(19) United States
(12) Patent Application Publication
(10) Pub. No.: US 2024/0174792 A1
(43) Pub. Date: May 30, 2024

(54) POLYMER

(71) Applicant: Sumitomo Chemical Co., Ltd., Tokyo (JP)

(72) Inventor: Nazrul Islam, Godmanchester (GB)

(73) Assignee: Sumitomo Chemical Co., Ltd., Tokyo (JP)

(21) Appl. No.: 18/281,283

(22) PCT Filed: Mar. 11, 2022
(86) PCT No.: PCT/EP2022/056378

§ 371 Date: Sep. 8, 2023

(30) Foreign Application Priority Data
Mar. 11, 2021 (GB) 2103379.0

(51) Int. Cl. C08G 61/12; C07F 5/02; C09K 11/06; C12Q 1/6869; G01N 33/58

(52) U.S. Cl. CPC C08G 61/124; C07F 5/027; C09K 11/06; C12Q 1/6869; G01N 33/582; C08G 2261/122; C08G 2261/1424; C08G 2261/1426; C08G 2261/148; C08G 2261/18; C08G 2261/3142; C08G 2261/316; C08G 2261/3247; C08G 2261/411; C08G 2261/52; C09K 2211/1416; C09K 2211/1425; C09K 2211/1433; C09K 2211/1491

(57) ABSTRACT

A conjugated polymer comprising a repeat unit of formula (I) wherein R¹ in each occurrence is a substituent; R² in each occurrence is independently H or a substituent; X is selected from O, S and NR³; and R³ in each occurrence is independently H or a substituent. The polymer may be used as a light-emitting marker in a method of sequencing nucleic acids. The polymer may be used as a method of identifying a target analyte in a sample.

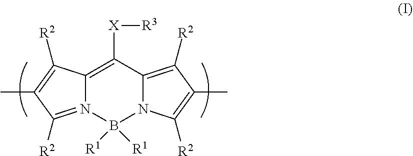

### BACKGROUND

The present disclosure relates to conjugated light-emitting polymers and their use as light-emitting markers.

U.S. Pat. No. 9,623,123 discloses a fluorescent conjugated polymer of formula (I):

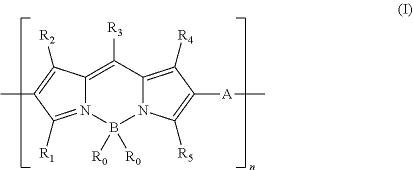

Kim, D., Ma, D., Kim, M. et al. Fluorescent Labeling of Protein Using Blue-Emitting 8-Amino-BODIPY Derivatives. J Fluoresc 27, 2231-2238 (2017) discloses blue fluorescence of 8-Amino-BODIPY compounds for protein labelling.

Bacalum et al, “A Blue-Light-Emitting BODIPY Probe for Lipid Membranes” Langmuir 2016, 32, 3495-3505 discloses a BODIPY-based membrane probe of formula:

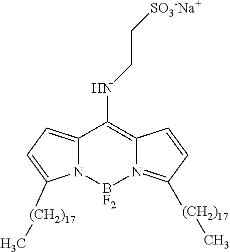

### SUMMARY

In some embodiments, the present disclosure provides a conjugated polymer comprising a repeat unit of formula (I):

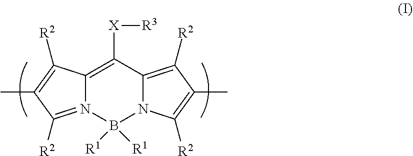

wherein:
R¹ in each occurrence is a substituent;
R² in each occurrence is independently H or a substituent;
X is selected from O, S and NR³; and
R³ in each occurrence is independently H or a substituent.

Optionally, each R³ is independently selected from;
non-ionic groups selected from:
   H;
   C₁₋₂₀ alkyl or C₁₋₂₀ alkylene-Ar² wherein Ar² is an aryl or heteroaryl group which may be unsubstituted or substituted with one or more non-ionic substituents and one or more non-adjacent C atoms of the C₁₋₂₀ alkyl or alkylene other than a terminal C atom or a C atom bound to X may be replaced with O, S, CO or COO and one or more H atoms may be replaced with F; and
   an aryl or heteroaryl group Ar² which is unsubstituted or substituted with one or more non-ionic substituents;
and
ionic groups.

Optionally, each R² is H.

Optionally, each R¹ is selected from F, CN, C₁₋₆ alkyl, C₁₋₆ alkynyl, —(OC2H₅)u-H wherein u is 1-5 and an unsubstituted or substituted aromatic or heteroaromatic group. R¹ is preferably F.

Optionally, X is NR³.

Optionally, the conjugated polymer is a copolymer comprising the repeat unit of formula (I) and one or more co-repeat units.

Optionally, the conjugated polymer comprises an arylene or heteroarylene co-repeat unit which is unsubstituted or substituted with one or more substituents.

Optionally, the conjugated polymer has a photoluminescence spectrum having a peak in the range of 400-500 nm. Optionally, the emission peak of the photoluminescence spectrum has a full width at half maximum (FWHM) of no more than 75 nm.

Optionally, the conjugated polymer has a Stokes shift of less than 50 nm.

Optionally, the peak of an absorption spectrum of the conjugated polymer has a FWHM of no more than 50 nm

Optionally, the polymer has a solubility of at least 0.1 mg/mg in at least one of water and methanol.

In some embodiments, the present disclosure provides a monomer of formula (I-M):

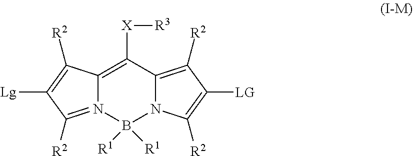

wherein:
R¹ in each occurrence is a substituent;
R² in each occurrence is independently H or a substituent;
X is selected from O, S and NR³;
R³ in each occurrence is independently H or a substituent; and
Each LG is independently selected from the group consisting of boronic acid; boronic esters; halides; and pseudohalides.

In some embodiments, the present disclosure provides a method of forming a conjugated polymer as described herein, comprising polymerisation of a monomer as described herein.

In some embodiments, the present disclosure provides a light-emitting particle comprising the conjugated polymer as described herein.

Optionally, the particle comprises a matrix material.

Optionally, the matrix material is silica.

In some embodiments, the present disclosure provides a light-emitting marker comprising the conjugated polymer or the light-emitting particle as described herein and a binding group comprising a biomolecule.

In some embodiments, the present disclosure provides a precursor of the light-emitting marker described herein comprising a functional group capable of attaching to the binding group.

In some embodiments, the present disclosure provides a method of forming the light-emitting marker as described herein comprising attaching the binding group to the functional group of the precursor as described herein.

In some embodiments, the present disclosure provides a formulation comprising the conjugated polymer, light-emitting particle or precursor as described herein dissolved or dispersed in one or more solvents.

In some embodiments, the present disclosure provides a method of sequencing nucleic acids comprising contacting a primed template nucleic acid molecule with a polymerase and a test nucleotide;
incorporating the test nucleotide into a primed strand of the primed template only if it comprises a base complementary to the next base of the template strand;
irradiating the primed strand; and
determining from luminance of the primed strand if the test nucleotide has been incorporated into the primed strand,
wherein the test nucleotide of the irradiated primed strand is bound to a conjugated polymer, light-emitting particle or light-emitting marker as described herein configured to bind to the test nucleotide.

In some embodiments, the present disclosure provides a method of identifying a target analyte in a sample, the method comprising irradiating the sample to which has been added a conjugated polymer, light-emitting particle or light-emitting marker as described herein configured to bind to the target analyte; and detecting emission from the conjugated polymer. Optionally, the target analyte is a cell and the method is a flow cytometry method.

### DESCRIPTION OF THE DRAWINGS

The disclosed technology and accompanying figures describe some implementations of the disclosed technology.

FIG. 1 is a graph of emission and absorption spectra for a light-emitting polymer according to some embodiments.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

In some embodiments, the present disclosure provides a conjugated polymer comprising a repeat unit of formula (I):

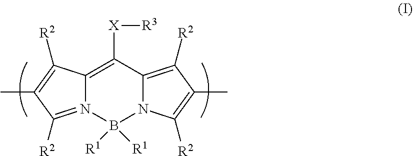

R¹ in each occurrence is a substituent, preferably F, CN, C₁₋₆ alkyl, C₁₋₆ alkynyl, —(OC2H₅)u-H wherein u is 1-5 and an aromatic or heteroaromatic group, preferably phenyl. An aromatic or heteroaromatic group R¹ may be unsubstituted or substituted with one or more substituents, optionally one or more substituents selected from F, CN, NO₂ and C₁₋₁₂ alkyl wherein one or more non-adjacent, non-terminal C atoms of a C₂₋₁₂ alkyl may be replaced with O, S, CO or COO and one or more H atoms may be replaced with F.

R² in each occurrence is independently H or a substituent, preferably H or C₁₋₆ alkyl.

X is selected from O, S and NR³.

R³ is H or a substituent. In the case where X is NR³, each R³ may be the same or different.

The present inventors have found that a conjugated polymer containing repeat units of formula (I) may provide emission in the blue (<500 nm) region of the electromagnetic spectrum. The present inventors have further found that polymers as described herein may have an absorption peak wavelength at wavelength greater than 400 nm. The polymer may therefore have a small (e.g. less than 50 nm) Stokes shift.

This small Stokes shift may be advantageous in assay methods for detection of a target analyte, e.g. a target cell, or in nucleic acid sequencing methods. For example, in a DNA sequencing method the use of a polymer as described herein as an emitter may allow for reduction in well size due to less diffraction as compared to an emissive species having a longer peak emission wavelength. Furthermore, the small Stokes shift may allow a sample to be irradiated with reduced damage to the DNA chain or nucleic acids in the sample as compared to use of an emitter having a larger Stokes shift, e.g. a blue emissive material having an absorption peak in the UV region at below 400 nm.

A conjugated polymer as described herein is a polymer having a backbone containing repeat units that are directly conjugated to one another. In some embodiments, the whole of the backbone is conjugated. In some embodiments, the backbone may comprise repeat units conjugated to one another and repeat units which interrupt conjugation along the polymer backbone. Preferably, the conjugated polymer comprises repeat units of formula (I) which are directly conjugated to adjacent repeat units.

Preferably, R³ is selected from:
non-ionic groups selected from:
   H;
   C₁₋₂₀ alkyl or C₁₋₂₀ alkylene-Ar² wherein Ar² is an aryl or heteroaryl group, preferably phenyl, which may be unsubstituted or substituted with one or more non-ionic substituents and one or more non-adjacent C atoms of the C₁₋₂₀ alkyl or alkylene other than a terminal C atom or a C atom bound to X may be replaced with O, S, CO or COO and one or more H atoms may be replaced with F; and
   an aryl or heteroaryl group Ar² which is unsubstituted or substituted with one or more non-ionic substituents;
and
ionic substituents.

By “terminal C atom” of an alkyl group as used herein means the methyl (—CH₃) group or groups, respectively, of a linear or branched a branched alkyl.

The one or more substituents of Ar², where present, are optionally and independently selected from F, CN, NO₂ and C₁₋₂₀ alkyl wherein one or more non-adjacent, non-terminal C atoms of the alkyl may be replaced with O, S, COO or CO and one or more H atoms of the alkyl may be replaced with F.

A preferred non-ionic substituent R³ comprises or consists of a group of formula (II):

—(CH₂)ₐ—(OC₂H₅)_{b}—OR⁴ (II)

wherein:
a is at least 1, optionally 1, 2, 3 or 4;
b is at least 1, preferably more than 1, e.g. 2-12;
and R⁴ is H or a C₁₋₅ alkyl group.

Preferably, R³ is H or a group which increases the solubility of the conjugated polymer in water as compared to the case where each R³ is H, for example a group of formula (II) or an ionic substituent.

Preferably, an ionic substituent as described herein has formula (III):

-(Sp¹)ₘ-(R⁵)ₙ (III)

wherein Sp¹ is a spacer group; m is 0 or 1; R⁵ independently in each occurrence is an ionic group; n is 1 if m is 0 and n is at least 1, optionally 1, 2, 3 or 4, if m is 1.

Preferably, Sp¹ is selected from formulae (IVa), (IVb) or (IVc):

-Ak¹ (IVa)

-Ak¹-(Ar¹)_{b} (IVb)

-(Ar¹)_{b} (IVc)

wherein Ak¹ is a C₁₋₂₀ alkylene wherein one or more non-adjacent, non-terminal C atoms other the C atom bound X may be replaced with O, S, or C═O or COO;

Ar¹ in each occurrence is independently an arylene or heteroarylene group, preferably phenylene, which, in addition to one or more ionic groups R⁵, may be unsubstituted or substituted with one or more non-ionic substituents; and

b is at least 1, optionally 1-3, preferably 1.

Non-ionic substituents of Ar¹ may be selected from F; CN; NO₂; and C₁₋₁₂ alkyl wherein one or more non-adjacent, non-terminal C atoms may be replaced with O, S, C═O or COO and one or more H atoms may be replaced with F.

More preferably, Sp¹ is selected from:
C₁₋₂₀ alkylene wherein one or more non-adjacent C atoms other than the C atom bound to X are replaced with O;
C₁₋₁₂ alkylene-Ar¹, more preferably C₁₋₁₂ alkylene-phenylene, wherein one or more non-adjacent C atoms of the alkylene may be replaced with O and the Ar¹ group is, in addition to one or more ionic groups R⁵, unsubstituted or substituted with one or more non-ionic substituents; and
a C₆₋₂₀ arylene or a 5-20 membered heteroarylene, even more preferably phenylene, which is, in addition to one or more ionic groups R⁵, unsubstituted or substituted with one or more non-ionic substituents.

In a preferred embodiment, Sp¹ is phenylene or C₁₋₁₂ alkylene-phenylene, wherein the phenylene is substituted with at least one ionic group R⁵ and at least one group of formula (V):

—O(R⁶O)ᵥ-R⁷ (V)

wherein R⁶ in each occurrence is a C₁₋₁₀ alkylene group, optionally a C₁₋₅ alkylene group; R⁷ is H or C₁₋₅ alkyl; and v is 0 or a positive integer, optionally 1-10. Preferably, v is at least 2. More preferably, v is 2 to 5. The value of v may be the same in all the polar groups of formula (V). The value of v may differ between different groups of formula (V) of the same polymer.

Optionally, the group of formula (V) has formula —O(CH₂CH₂O)ᵥR⁷ wherein v is at least 1, optionally 1-10 and R⁷ is a C₁₋₅ alkyl group, preferably methyl. Preferably, v is at least 2. More preferably, v is 2 to 10.

The ionic group R⁵ may be anionic or cationic.

Exemplary anionic group are —COO⁻, a sulfonate group; hydroxide; sulfate; phosphate; phosphinate; or phosphonate.

An exemplary cationic group is —N(R⁸)₃⁺ wherein R⁸ in each occurrence is H or C₁₋₂₀ hydrocarbyl. Preferably, each R⁸ is a C₁₋₂₀ hydrocarbyl.

A C₁₋₂₀ hydrocarbyl group as described anywhere herein is optionally selected from a linear, branched or cyclic alkyl, optionally C₁₋₂₀ alkyl; unsubstituted phenyl; and phenyl substituted with one or more C₁₋₁₂ alkyl groups.

A conjugated polymer as described herein comprising cationic or anionic groups comprises counterions to balance the charge of these ionic groups. An anionic or cationic group and counterion may have the same valency, with a counterion balancing the charge of each anionic or cationic group. The anionic or cationic group may be monovalent or polyvalent. Preferably, the anionic and cationic groups are monovalent.

The conjugated polymer may comprise a plurality of anionic or cationic polar groups wherein the charge of two or more anionic or cationic groups is balanced by a single counterion. Optionally, the polar groups comprise anionic or cationic groups comprising di- or trivalent counterions.

In the case of an anionic group, the cation counterion is optionally a metal cation, optionally Li⁺, Na⁺, K⁺, Cs⁺, preferably Cs⁺, or an organic cation, optionally ammonium, such as tetraalkylammonium, ethylmethyl imidazolium or pyridinium.

In the case of a cationic group, the anion counterion is optionally a halide; a sulfonate group, optionally mesylate or tosylate; hydroxide; carboxylate; sulfate; phosphate; phosphinate; phosphonate; or borate.

Exemplary repeat units of formula (I) include, without limitation:

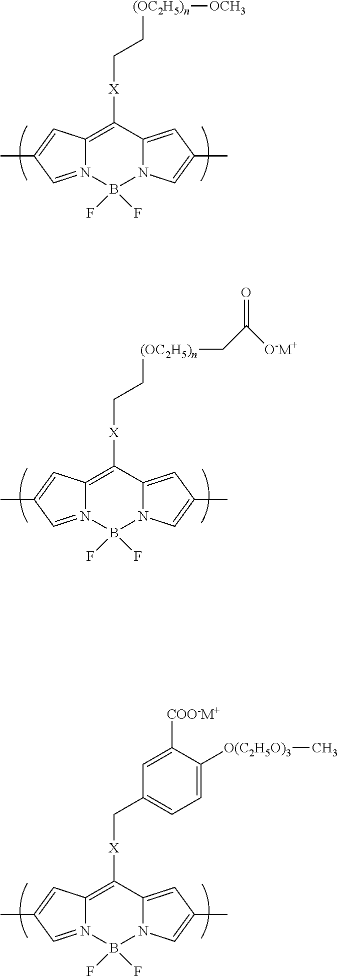

The polymer may have a solubility in at least one of water and a C₁₋₈ alcohol at 20° C. of at least 0.1 mg/ml, optionally at least 0.5 mg/ml or at least 1 mg/ml.

The polymer may have a solubility in a C₁₋₄ alcohol, preferably methanol, at 20° C. of at least 0.1 mg/ml, optionally at least 0.5 mg/ml or at least 1 mg/ml.

Solubility may be measured by the following method:

The solid polymer is weighed out into a glass vial. The required amount of solvent is added followed by a small magnetic stirrer. Then the vial is tightly capped and put on a preheated hot plate at 60° C. with stirring for 30 min. The polymer solution is allowed to cool to room temperature before use. The polymer solution can also be prepared by sonicating the polymer containing vial for 30 min at room temperature. The solubility of polymer was tested by visual observation and under white and 365 nm UV light.

### Co-Repeat Units

The polymer described herein may be a homopolymer in which all repeat units are units of formula (I).

Preferably, the polymer described herein is a copolymer comprising a repeat unit of formula (I) and one or more co-repeat units. Repeat units may make up 1-99 mol % of the repeat units of a polymer, optionally 3-80 mol % or 5-50 mol % of the repeat units of a polymer. Exemplary co-repeat units include arylene co-repeat units which may be unsubstituted or substituted with one or more substituents; heteroarylene co-repeat units which may be unsubstituted or substituted with one or more substituents; and conjugation-breaking repeat units.

Exemplary arylene and heteroarylene co-repeat units include repeat units of formulae (VI)-(XII):

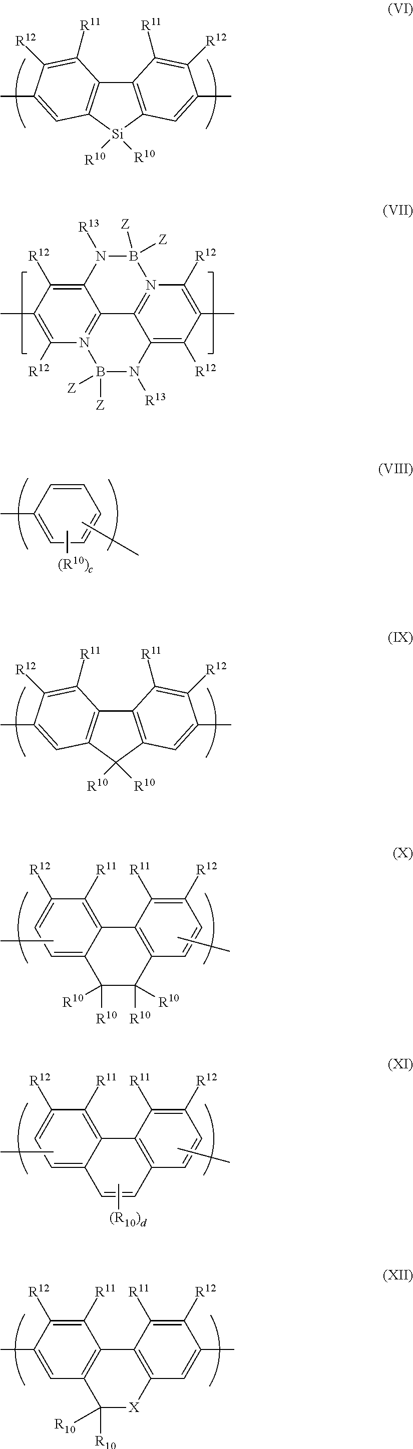

wherein:
R¹⁰ in each occurrence is independently a substituent;
R¹¹ in each occurrence is independently H or a substituent R¹⁰ and two R¹¹ groups may be linked to form a ring;
R¹² independently in each occurrence is H or a substituent;
R¹³ independently in each occurrence is a C₁₋₂₀ hydrocarbyl group;
Z in each occurrence is independently a substituent, preferably F or a C₁₋₂₀ hydrocarbyl group;
c is 0, 1, 2, 3 or 4, preferably 1 or 2; and
d is 0, 1 or 2.

Optionally, each R¹⁰ is independently selected from:
C₁₋₂₀ alkyl wherein one or more non-adjacent, non-terminal C atoms may be replaced with O, S, COO or CO and one or more H atoms of the alkyl may be replaced with F; p1 a group of formula —(Ar³) p wherein Ar³ in each occurrence is independently an aryl or heteroaryl group, preferably phenyl, which is unsubstituted or substituted with one or more substituents and p is at least 1, optionally 1, 2 or 3; and
an ionic substituent R⁵.

Preferably, each R¹¹ is H or both R¹¹ groups are linked to form a ring, optionally a 6 or 7 membered ring. Optionally, two R¹¹ groups are linked to form a ring in which the linked R¹¹ groups form a C₂- or C₃-alkylene chain wherein one or more non-adjacent C atoms of the alkylene chain may be replaced with O, S, NR¹³ or Si(R¹³)₂ wherein R¹³ in each occurrence is independently a C₁₋₂₀ hydrocarbyl group.

An exemplary repeat unit in which both R¹¹ groups are linked has formula (IXa):

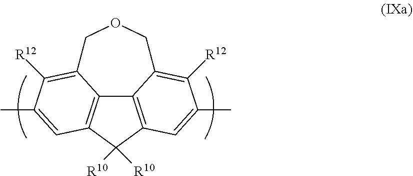

Each R¹² is preferably H or a substituent R¹⁰, more preferably H.

Where present, repeat units selected from formulae (VI)-(XII) make up 1-99 mol % of the repeat units of the polymer, preferably at least 25 mol % or at least 50 mol % of the repeat units of the polymer.

The conjugation-breaking repeat unit may have formula (XV):

[symbol]

Ar⁴-CB-Ar⁴ [symbol] (XV)

wherein Ar⁴ and Ar⁵ each independently represent a C₆₋₂₀ arylene group or a 5-20 membered heteroarylene group which is unsubstituted or substituted with one or more substituents; and CB represents a conjugation-breaking group which does not provide a conjugation path between Ar⁴ and Ar⁵.

Where present, the repeat unit of formula (XV) makes up 1-50 mol %, optionally 1-25 mol %, of the repeat units of the polymer.

Ar⁴ and A⁵ are each independently unsubstituted or substituted with one or more substituents. Substituents of Ar⁴ and A⁵, where present, are optionally selected from substituents R¹⁰ as described above.

Optionally, Ar⁴ and A⁵ are each independently unsubstituted or substituted phenylene, optionally 1,3- or 1,4-linked phenylene.

CB does not provide any conjugation path between Ar⁴ and A⁵. Optionally, CB does not provide a path of alternating single and double bonds between Ar⁴ and Ar⁵.

Optionally, CB is a C₁₋₂₀ branched or linear alkylene group wherein one or more H atoms may be replaced with F and one or more non-adjacent C atoms of the alkylene group may be replaced with O, S, CO, COO or Si(R¹⁶)₂ wherein R¹⁶ in each occurrence is independently a C₁₋₂₀ hydrocarbyl group.

Optionally, CB contains least one sp³ hybridised carbon atom separating Ar⁴ and A⁵.

The conjugation-breaking repeat unit may have formula (XVa) or (XVb):

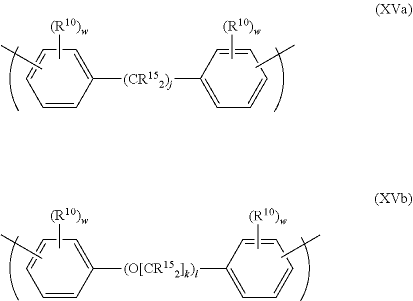

wherein each w is independently 0-4, optionally 0, 1 or 2; each R¹⁰ is independently a substituent as described above; each R¹⁵ is independently H or a C₁₋₆ alkyl group, preferably H; j is at least 1; k is at least 1; and 1 is at least 1.

In some embodiments, each w is 0.

In some embodiments, at least one w is 1 or 2.

Optionally, j is 2-20 or 2-12.

Optionally, k is 2-6, preferably 2.

Optionally, 1 is 1-6.

In a preferred embodiment, the conjugated polymer comprises a co-repeat unit substituted with at least one ionic substituent, preferably an ionic substituent R⁵ as described herein. More preferably, the co-repeat unit substituted with at least one ionic substituent is selected from co-repeat units of formulae (VI)-(XII). Exemplary repeat units substituted with an ionic substituent include, without limitation:

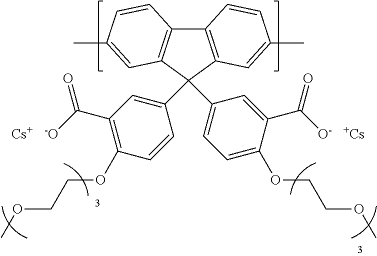

### Polymer Formation

The conjugated light-emitting polymers described herein may be formed by any method known to the skilled person. Arrangement of repeat units within the polymer backbone may be controlled by, e.g. formation of block copolymers, use of polymerisation methods requiring monomers with different reactive groups; and selection of monomer ratio.

Conjugated polymers as described herein may be formed by polymerising monomers comprising leaving groups that leave upon polymerisation of the monomers to form conjugated repeat units. Exemplary polymerization methods include, without limitation, Yamamoto polymerization as described in, for example, T. Yamamoto, “Electrically Conducting And Thermally Stable pi-Conjugated Poly(arylene)s Prepared by Organometallic Processes”, Progress in Polymer Science 1993, 17, 1153-1205, the contents of which are incorporated herein by reference and Suzuki polymerization as described in, for example, WO 00/53656, WO 2003/035796, and U.S. Pat. No. 5,777,070, the contents of which are incorporated herein by reference.

The monomers may be formed by polymerisation of monomers containing boronic acid leaving groups or esters thereof, and halide or pseudohalide (e.g. sulfonate) leaving groups. Optionally, the polymer is formed by polymerisation of a monomer of formula (I-M):

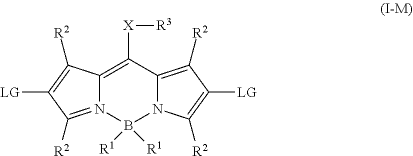

wherein LG is a boronic acid, boronic ester, halide or pseudohalide leaving group.

### Nucleic Acid Sequencing

A light-emitting marker comprising or consisting of a conjugated polymer as described herein may be used as a in a method for analysing and/or sequencing nucleic acids in which the light-emitting marker is bound, suitably covalently bound, to a nucleotide.

In some embodiments of this method, a primed template nucleic acid molecule is contacted a polymerase and a test nucleotide. The test nucleotide is incorporated into the primed strand of the primed template only if it comprises a base complementary to the next base of the template strand. Emission of light from the conjugated polymer bound to the nucleotide is indicative of incorporation of the test nucleotide into the primed strand.

In some embodiments, the conjugated polymer is bound to the test nucleotide before it is brought into contact with the polymerase and the primed template nucleic acid molecule. In some embodiments, the light-emitting polymer binds to the test nucleotide after it has been incorporated into the primed strand.

The conjugated polymer may be substituted with a binding group which binds to the test nucleotide. The test nucleotide may be substituted with a complementary group for binding to the binding group. For example, one of the test nucleotide and the conjugated polymer may be functionalised with biotin and the other of the test nucleotide and the conjugated polymer may be functionalised with avidin, streptavidin, neutravidin or a recombinant variant thereof.

In some embodiments, the conjugated polymer is bound to the test nucleotide by a cleavable linker, e.g. a cleavable linker formed by binding of the functional groups. Following detection of any emission from the conjugated polymer, the linker may be cleaved to separate the polymer from the primed strand and the primed strand may then be brought into contact with a further test nucleotide. Cleavage may be by treatment with a cleaving agent. Cleavage may be by irradiation. Exemplary cleavable linkers are disclosed in Leriche et al, “Cleavable linkers in chemical biology”, Bioorganic & Medicinal Chemistry, Vol. 20, Issue 2, 2012, p. 571-582, the contents of which are incorporated herein in its entirety and include, without limitation, carbamates; and groups that may be cleaved by a transition metal or a phosphine catalyst such as ethers having a disulfide alpha substituent and ethers having an azide alpha substituent.

### Assay Methods

Optionally the light-emitting marker is for use in fluorescence microscopy, flow cytometry, luminescent imaging applications for example in-vivo imaging, or any other application where a light-emitting marker is brought into contact with a sample to be analysed. The analysis may be performed using time-resolved spectroscopy. The applications can medical, veterinary, agricultural or environmental applications whether involving patients (where applicable) or for research purposes.

Preferably, the light-emitting marker is excited by irradiation by light having a wavelength greater than 400 nm, optionally greater than 400 nm up to 450 nm. Preferably, emission from the light-emitting marker is detected by a photodetector configured to detect emission at a wavelength of less than 500 nm, preferably in the range of 450-500 nm.

In use the binding group of the light-emitting markers may bind to target biomolecules which include without limitation DNA, RNA, peptides, carbohydrates, antibodies, antigens, enzymes, proteins and hormones. The target biomolecule may or may not be a biomolecule, e.g. a protein, at a surface of a cell.

A sample to be analysed may brought into contact with the light-emitting marker, for example the light-emitting marker dissolved in a solution or a particulate light-emitting marker in a colloidal suspension.

In some embodiments, the sample is analysed by flow cytometry. In flow cytometry, the light-emitting marker or markers are irradiated by at least one wavelength of light, optionally two or more different wavelengths, e.g. one or more wavelengths including at least one of about 355, 405, 488, 530, 561 and 640 nm, each of which may be ±10 nm. Light emitted by the light-emitting marker(s) may be collected by one or more detectors. To provide a background signal for calculation of a staining index, measurement may be made of a light-emitting marker mixed with cells which do not bind to the light-emitting marker.

A conjugated polymer as described herein may be used as a light-emitting marker in methods for detection of a target analyte.

In some embodiments, a binding group having affinity for a target analyte is bound, preferably covalently bound, to the conjugated light-emitting polymer. The binding group may be provided as a side group of a repeat unit of the light-emitting polymer or as an end-group of the light-emitting polymer. In some embodiments, the conjugated light-emitting polymer in use, e.g. in flow cytometry, may be dissolved or dispersed in a sample to be analysed. In the case where it is dissolved, the conjugated light-emitting polymer is preferably dissolved in water.

### Light-Emitting Particle

A particulate light-emitting marker may comprise or consist of a conjugated polymer as described herein.

In some embodiments, the light-emitting marker particles comprise the conjugated light-emitting polymer in collapsed form.

In preferred embodiments, the light-emitting marker particles comprise a matrix material and the conjugated light-emitting polymer. The matrix material is preferably an inorganic matrix material, e.g. silica. According to these embodiments, the binding group may be bound, preferably covalently bound, to the matrix.

Matrix materials include, without limitation, inorganic matrix materials, optionally inorganic oxides, optionally silica. The matrix may at least partially isolate the light-emitting material from the surrounding environment. This may limit any effect that the external environment may have on the lifetime of the light-emitting material.

Light-emitting marker particles may comprise a core and, optionally, one or more shells surrounding the core.

Polymer chains of the conjugated light-emitting polymer may extend across some or all of the thickness of the core and/or shell. Polymer chains may be contained within the core and/or shell or may protrude through the surface of the core and/or shell.

The conjugated light-emitting polymer may be mixed with the matrix material.

The conjugated light-emitting polymer may be bound, e.g. covalently bound, to the matrix material.

In some embodiments, the particle core may be formed by polymerisation of a silica monomer in the presence of the light-emitting polymer, for example as described in WO 2018/060722, the contents of which are incorporated herein by reference.

In some embodiments, the particle core comprises a core which comprises or consists of the light-emitting polymer and at least one shell surrounding the inner core. The at least one shell may be silica.

Optionally, at least 0.1 wt % of total weight of the particle core consists of the conjugated light-emitting polymer. Preferably at least 1, 10, 25 wt % of the total weight of the particle core consists of the conjugated light-emitting polymer.

Optionally at least 50 wt % of the total weight of the particle core consists of the matrix material. Preferably at least 60, 70, 80, 90, 95, 98, 99, 99.5, 99.9 wt % of the total weight of the particle core consists of the matrix material.

The particle core as described herein is the light-emitting particle without any surface groups, e.g. binding groups or solubilising groups, thereon.

In one embodiment of the present disclosure, at least 70 wt % of the total weight of the particle core consists of the conjugated light-emitting polymer and silica. Preferably at least 80, 90, 95, 98, 99, 99.5, 99.9 wt % of the total weight of the particle core consists of the conjugated light-emitting polymer and silica. More preferably the particle core consists essentially of the conjugated light-emitting polymer and silica.

Preferably, the particles have a number average diameter of no more than 5000 nm, more preferably no more than 2500 nm, 1000 nm, 900 nm, 800 nm, 700 nm, 600 nm, 500 nm or 400 nm as measured by dynamic light scattering (DLS) using a Malvern Zetasizer Nano ZS. Preferably the particles have a number average diameter of between 5-5000 nm, optionally 10-1000 nm, preferably between 10-500 nm, most preferably between 10-100 nm as measured by a Malvern Zetasizer Nano ZS.

Surface groups may be bound to a surface of the light-emitting particles. Exemplary surface groups include, without limitation, ether-containing groups, e.g. groups containing poly(ethyleneglycol) (PEG) chains and groups containing a binding group comprising a biomolecule.

Light-emitting particles as described herein may be provided as a colloidal suspension comprising the particles suspended in a liquid. Preferably, the liquid is selected from water, C₁₋₁₀ alcohols and mixtures thereof. Preferably, the particles form a uniform (non-aggregated) colloid in the liquid. In some embodiments, each of the first, second and any further light-emitting markers are light-emitting particles dispersed in the liquid. In some embodiments, one or more of the light-emitting markers is in particle form dispersed in the liquid and one or more of the light-emitting markers is dissolved in the liquid.

The liquid may be a solution comprising salts dissolved therein, optionally a buffer solution.

In some embodiments, the particles may be stored in a powder form, optionally in a lyophilised or frozen form.

### Functional Groups

A binding group of the light-emitting marker, e.g. for binding to a target analyte or a test nucleotide, may be attached to the light-emitting marker by attachment to a functional group of a precursor of the light-emitting marker.

In some embodiments, the functional group is covalently bound to the conjugated light-emitting polymer.

In some embodiments, the functional group is covalently bound to a matrix material of a particulate marker precursor comprising the matrix material and the conjugated light-emitting polymer.

Optionally the functional group is selected from:
amine groups, optionally —NR⁹₂ wherein R⁹ in each occurrence is independently H or a substituent, preferably H or a C₁₋₅ alkyl, more preferably H;
carboxylic acid or a derivative thereof, for example an anhydride, acid chloride or ester, acid chloride, acid anhydride or amide group;
—OH; —SH; an alkene; an alkyne; and an azide; and
biotin or a biotin-protein conjugate.

The functional group may be reacted with a biomolecule to form a linking group linking the biomolecule to the rest of the light-emitting marker, the linking group being selected from esters, amides, urea, thiourea, Schiff bases, a primary amine (C—N) bond, a maleimide-thiol adduct or a triazole formed by the cycloaddition of an azide and an alkyne.

Exemplary binding group biomolecules for binding to a target analyte include, without limitation, DNA, RNA, peptides, carbohydrates, antibodies, antigens, enzymes, proteins, hormones and combinations thereof.

In the case where the functional group is biotin, it may be conjugated to a protein, e.g. avidin, streptavidin, neutravidin and recombinant variants thereof, and a biotinylated biomolecule may be conjugated to the protein to form the light-emitting marker.

The biotinylated biomolecule may comprise an antigen binding fragment, e.g. an antibody, which may be selected according to a target antigen.

In the case of a light-emitting particle, the functional group may be bound to a surface of the light-emitting particle, e.g. bound to a matrix material of the light-emitting particle. Each functional group may be directly bound to the surface of a light-emitting particle or may be spaced apart therefrom by one or more surface binding groups. The surface binding group may comprise polar groups. Optionally, the surface binding group comprises a polyether chain. By “polyether chain” as used herein is meant a chain having two or more ether oxygen atoms.

The surface of a light-emitting particle core may be reacted to form a group at the surface capable of attaching to a functional group. Optionally, a silica-containing particle is reacted with a siloxane.

### Applications

Light-emitting markers as described herein may be used as luminescent probes for detecting or labelling a biomolecule or a cell. In some embodiments, the particles may be used as a luminescent probe in an immunoassay such as a lateral flow or solid-state immunoassay.

### Measurements

Unless stated otherwise, emission spectra are as measured in methanol in the case of a dissolved polymer or in water in the case of a particle containing a polymer as described herein, using a Hamamatsu C9920-02 instrument having a set up wavelength 300 nm-950 nm; light source 150 W xenon light and bandwidth 10 nm or less (FWHM). Initially the system was calibrated with red (395 nm), green (375 nm) and blue (335 nm) glass standards. Two 5 mL long necked cuvettes (one filled with reference solvent e.g. methanol or water) and one filled with a sample of 1 mg/mL diluted 1 in 100 for a dissolved light-emitting marker or 1 mg/ml diluted ˜1 in 10 with water for a particulate light-emitting marker. The final concentration of the sample was altered to obtain a transmission data in the range 0.25-0.35. An average of 3 measurements for each sample is recorded.

Unless stated otherwise, absorption spectra of a polymer as described herein are measured in methanol using a Cary 5000 UV-VIS-NIR Spectrometer. Absorption and emission spectra of a particulate light-emitting material are measured as a dispersion in water. Measurements were taken from 175 nm to 3300 nm using a PbSmart NIR detector for extended photometric range with variable slit widths (down to 0.01 nm) for optimum control over data resolution. A baseline run with water in front and back 5 mL matched cuvettes (600 to 250 nm) following which the back cuvette reference remained as methanol and the front cuvette was changed to a sample of 1 mg/ml diluted 1 in 100 for a dissolved light-emitting marker or 1 mg/mL diluted ˜1 in 10 with water for a particulate light-emitting marker.

### EXAMPLES

Monomer Example 1 was prepared according to reaction scheme 1.

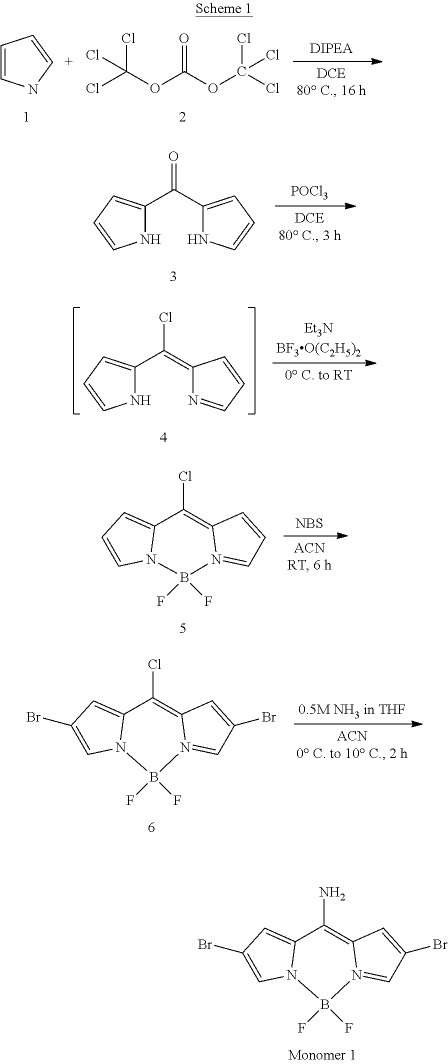

### Synthesis of Intermediate 3

| | Quantity | Vol. | | | |
|---|---|---|---|---|---|
| Reagent | (g) | (mL) | M.W | Moles | Eq. |
| 1H pyrrole | — | 68 | 67.09 | 0.9801 | 8.24 |
| Triphosgene | 35.3 | — | 296.75 | 0.1189 | 1 |
| N,N-Diisopropylethylamine | — | 58.3 | 129.24 | 0.3343 | 2.8 |
| 1,2-dichloroethane | — | 500 | — | — | — |

The reaction was performed using a 1 L 4-necked round-bottomed flask equipped with a mechanical stirrer, reflux condenser, nitrogen inlet and exhaust. 1H-pyrrole (25 mL), N-diisopropylethylamine (58.3 mL, 0.3343 mol) and 1,2-dichloroethane (150 mL) were added to in a 1 L round bottom flask. The flask was cooled to 0° C. and triphosgene (35.3 g, 0.119 mol) dissolved in 1,2-dichloroethane (350 mL) was slowly added into the reaction mixture. The above reaction mixture was stirred at 0° C. for 2 h and monitored by TLC using 20% ethyl acetate: DCM by which a polar spot and absence of starting material (visualized by using KMnO₄ stain) was observed. Again 1H-pyrrole (25 mL) was introduced slowly and heated at 80° C. for an hour, and reaction monitoring by TLC showed 70% unreacted starting material. Once again 1H-pyrrole (18 mL) was added drop wise at 80° C. and stirring continued at 80° C. for overnight. The two batch reaction mixtures were cooled to room temperature (25° C.), combined and diluted with MTBE (3 L) and poured on to water (3 L). The above mixture was filtered through a Buchner funnel and solids separated out. The layers of the filtrate were separated and the organic layer was washed with water and saturated brine solution, dried over anhydrous sodium sulphate and concentrated to obtain the crude product (47 g). The crude product was purified by column chromatography in 3 lots (15.6 g each) using 30% ethyl acetate in hexane as eluent to give 10 g of 92% LCMS purity. The obtained solid was recrystallized with 2% ethyl acetate in pet ether (200 mL) at room temperature (25° C.) for 2 hours and filtered through Buchner funnel to obtain 7.89% of 99.68% LCMS purity. Yield=21%. ¹H-NMR (400 MHz, CDCl₃): δ [ppm] 6.37-6.38 (m, 2H), 7.10-7.10 (m, 2H), 7.17-7.17 (m, 2H), 9.62 (bs, 2H).

### Synthesis of Intermediate 5

| | Quantity | Vol. | | | |
|---|---|---|---|---|---|
| Reagent | (g) | (mL) | MW | Moles | Eq. |
| Intermediate 3 | 8 | — | 160.17 | 0.0187 | 1 |
| Phosphorous oxychloride | — | 9.32 | 153.33 | 0.0997 | 5.3 |
| Triethyl amine | — | 70 | 101.19 | 0.5271 | 28 |
| Boron trifluoride diethyl | — | 67.5 | 141.93 | 0.544 | 29.09 |
| etherate | | | | | |
| Dichloroethane | — | 120 | — | — | — |

A 250 mL 3-necked round-bottomed flask, equipped with a magnetic stirrer, nitrogen inlet and exhaust were used for the reaction. Intermediate 3 (8 g, 0.0187 mol) and dichloroethane (120 mL) was added to the 250 mL round-bottomed flask under nitrogen at room temperature (25° C.). To the above reaction mixture phosphorus oxychloride (9.32 mL, 0.0997 mol) was added slowly dropwise at room temperature (25° C.) and heated to 80° C. for 3 hours. Then reaction mass was cooled to 0° C. and triethylamine was added (70 mL, 0.5271 mol) followed by boron trifluoride diethyl etherate (67.5 mL, 0.544 mol), then the reaction mixture was allowed to attain room temperature (25° C.) and stirred for an hour. The progress of the reaction was monitored by TLC and LCMS. When LCMS showed 94% of product formation and absence of starting material, the reaction mixture was diluted with ether (300 mL), washed with water (500 mL) and saturated brine solution, dried over anhydrous sodium sulphate and concentrated under vacuum to give 9 g of crude product. The crude product was purified by column chromatography using ˜35% DCM in hexane as eluent to obtain 6 g of 99.7% LCMS purity. Yield=53%. ¹H-NMR (400 MHz, CDCl₃): δ [ppm] 6.60 (d, J=4.40 Hz, 2H), 7.43 (d, J=4.40 Hz, 2H), 7.91 (s, 2H).

### Synthesis of Intermediate 6

| | Qty | Vol. | | | |
|---|---|---|---|---|---|
| Reagent | (g) | (mL) | MW | Moles | Eq. |
| Intermediate 5 | 2.5 | — | 226.42 | 0.011 | 1 |
| NBS | 4.09 | — | 177.98 | 0.0229 | 2.1 |
| Acetonitrile | — | 310 | — | — | — |

A 500 mL 3-necked round-bottomed flask equipped with a mechanical stirrer, addition funnel, nitrogen inlet and exhaust were used for the reaction. Note: Two batches were made (2.5 g and 2 g), and at the column purification stage the fractions were combined for further purification by hot acetonitrile. Intermediate 5 (2.5 g, 0.011 mol) was combined with acetonitrile (250 mL) at room temperature (25° C.). NBS (4.09 g, 0.0229 mol) dissolved in acetonitrile (60 mL) was slowly added dropwise over a period of 20 minutes at room temperature (25° C.). The reaction was stirred for 6 hours at room temperature (25° C.). The above reaction mixture was diluted with ethyl acetate, then washed with water, saturated brine solution, dried over anhydrous sodium sulphate and concentrated under vacuum to get crude product of 70% LCMS purity. The crude product was purified by column chromatography using eluent ˜10% DCM in hexane to isolate 2 fractions. Fraction 1: 1.5 g of 75% LCMS purity. Fraction 2: 0.7 g of 93% LCMS purity. These fractions were combined with another 2 g batch fractions. Fraction 1: 1.2 g of 85% LCMS purity. Fraction 2: 0.5 g of 79% LCMS purity. All the above fractions were treated with hot acetonitrile in which the compound was combined with acetonitrile (60 mL;) and heated to 60° C. and slowly allowed to cool to room temperature, stirred for 2 hours, filtered and dried under vacuum to isolate 3.5 g of product. Yield=46%. ¹H-NMR (400 MHz, CDCl₃): δ [ppm] 7.45 (s, 2H), 7.82 (s, 2H).

### Synthesis of Monomer 1

| | Qty | Vol. | | | |
|---|---|---|---|---|---|
| Reagent | (g) | (mL) | MW | Moles | Eq. |
| Intermediate 6 | 2.4 | — | 384.21 | 0.0062 | 1 |
| Ammonia in THF (0.5M) | — | 36 | — | — | — |
| Acetonitrile | — | 240 | — | — | — |

A 500 mL multi neck RBF equipped with magnetic stir bar, condenser and N₂ inlet and exhaust and oil bath were used for the reaction. Note: Another 1 g batch reaction was performed, and the products of the batch reactions were combined at the purification stage. Intermediate 5 (2.4 g, 0.0062 mol) was combined with acetonitrile (240 mL) and cooled to 0° C., then a 0.5 M solution of ammonia in THF (36 mL) was slowly added dropwise. The reaction mixture was stirred for 2 hours at 0° C. to 10° C., after which reaction monitoring by LCMS showed 81.18% of desired product formation and absence of starting material. The reaction mixture was filtered through a celite plug and washed with a 1:1 mixture of DCM and methanol. The filtrate was concentrated to get 2.5 g of crude product. The crude product was purified by GRACE reverse phase column chromatography (C18 column) in 3 lots. Eluent: water/acetonitrile. At ˜56% acetonitrile in water, the desired product eluted. Note: Two batches of crude material were combined (2.5 g and 0.9 g) and purified using reverse phase column purification. Lot 1—Crude (1.1 g)→Fraction 1: 0.1 g of 77% LCMS purity along with monobromide impurity. Fraction 2: 0.47 g of 99.24% LCMS purity along with 0.6% tribromide impurity. Lot 2—Crude (1.1 g)→Fraction 1: 0.094 g of 68.5% LCMS purity along with 25.82% monobromide impurity. Fraction 2: 0.612 g of 98.59% LCMS purity along with 5.04% tribromide impurity. Fraction 3: 0.301 g of 93.32% LCMS purity along with 6.1% tribromide impurity. Lot 3—Crude (1.2 g)→Fraction 1: 0.722 g of 98.11% LCMS purity along with 3.61% tribromide impurity. Fraction 2: 0.071 g of 94.66% LCMS purity along with 4.4% tribromide impurity. Fraction 3: 0.301 g of 93.32% LCMS purity along with 6.1% tribromide impurity. Fractions of <98% purity were combined and re-purified by reverse phase column chromatography to obtain 1.3 g of 99.8% LCMS purity. All pure fractions were combined and stirred for 2 hours with n-hexane (40 mL, at room temperature (25° C.), filtered, and dried under vacuum to get 1.69 g of 99.65% HPLC purity as yellow colour solid. Yield=52%. ¹H-NMR (400 MHz, DMSO-d₆): δ [ppm] 7.57 (s, 4H), 9.97 (s, 2H).

The example Monomer 2 was synthesized according to scheme 2.

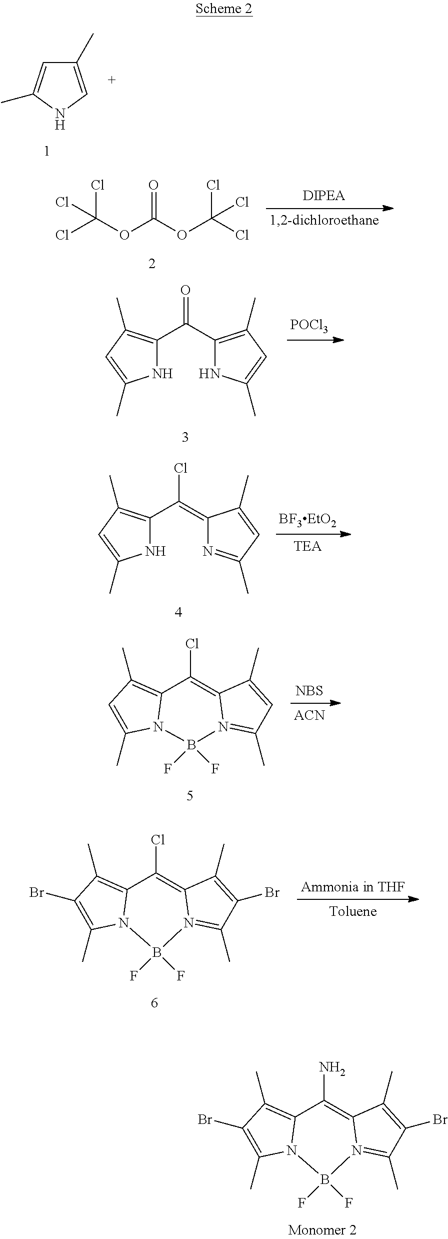

### Synthesis of Intermediate 3

| | | Vol. | | | |
|---|---|---|---|---|---|
| Reagent | Quantity(g) | (mL) | M.W | Moles | Eq. |
| 2,4-dimethyl-1H-pyrrole | 22.5 | 24.2 | 95.14 | 0.236 | 6.3 |
| Triphosgene | 11.1 | — | 296.75 | 0.0377 | 1 |
| N,N- | — | 18.4 | 129.24 | 0.103 | 2.73 |
| Diisopropylethylamine | | | | | |
| 1,2-dichloroethane | — | 225 | — | — | — |

A 500 mL×2 multi neck round-bottomed flask equipped with an additional funnel, magnetic stirring, condenser, oil bath N₂ inlet and exhaust were used for the reaction. Note: The reaction was performed in 22.5 g×2 parallel batches. Reaction mixtures were combined for isolation. 2,4-Dimethyl-/H-pyrrole (12.1 mL) and N-ethyl di-isopropylamine (18.4 mL, 103 mmol) and 1,2-dichloroethane (120 mL) were placed in the 500 mL round-bottomed flask. The flask was cooled to 0° C. and triphosgene (11.1 g, 37.7 mmol) dissolved in 1,2-dichloroethane (120 mL) was slowly added into the reaction mixture. The above reaction mixture was stirred at 0° C. for an hour. The reaction was monitored by TLC using 1:5 ethyl acetate: DCM by which a polar spot and absence of starting material (visualized by using KMnO₄ stain) was observed. Again 2,4-dimethyl-1H-pyrrole (12.1 mL) was introduced slowly and heated at 80° C. for 2 hours and the reaction was monitored by TLC for the desired product formation. Similarly, another reaction was carried out by following the above steps and the reaction mixtures were cooled to room temperature (25° C.), combined and diluted with MTBE (1 L) and water (500 mL). The organic layer was separated, the aqueous layer was extracted again with MTBE (300 mL), the combined organic layers were washed with water, saturated brine solution, dried over anhydrous sodium sulphate and concentrated to get crude product (37 g). The crude product was purified by column chromatography using an eluent of 20 to 25% ethyl acetate in hexane and two fractions were isolated. Fraction 1: 3.4 g with 94.63% LCMS purity. Fraction 2: 3.6 g with 96% LCMS purity. Both the fractions were combined and triturated with 10% ethyl acetate in petroleum ether (70 mL) at room temperature (25° C.) for 2 h, filtered and dried under vacuum to obtain 5.5 g of 98.4% LCMS purity. Yield=34%. ¹H-NMR (400 MHz, DMSO-d₆): δ [ppm] 2.03 (s, 6H), 2.17 (s, 6H), 5.75 (s, 2H), 10.91 (s, 2H).

### Synthesis of Intermediate 5

| | Quantity | Vol. | | | |
|---|---|---|---|---|---|
| Reagent | (g) | (mL) | MW | mmoles | Eq. |
| Intermediate 3 | 6 | — | 216.2 | 27.7 | 1 |
| Phosphorus oxychloride | — | 5.17 | 153.3 | 55.4 | 2 |
| Boron trifluoride diethyl | — | 37.4 | 141.9 | 304 | 11 |
| etherate | | | | | |
| Triethylamine | — | 38.7 | 101.1 | 276 | 10 |
| 1,2-dichloroethane | — | 100 | — | — | — |

A 500 mL 3-necked round-bottomed flask, equipped with a magnetic stirrer, nitrogen inlet and exhaust were used for the reaction. Intermediate 3 (6 g, 27.7 mmol) and dichloroethane (100 mL) were placed in the 500 mL RB flask under nitrogen at room temperature (25° C.). To the above reaction mixture phosphorus oxychloride (5.17 mL, 55.4 mmol) was added slowly dropwise at room temperature and heated to 80° C. for 3 h. The reaction mass was cooled to 0° C. and triethylamine (38.7 mL, 276 mmol) was slowly added dropwise using a dropping funnel followed by boron trifluoride diethyl etherate (37.4 mL, 304 mmol) and the reaction mixture was then allowed to attain room temperature (25° C.). The reaction mixture was stirred at room temperature for an hour and the progress of the reaction was monitored by TLC and LCMS.

When LCMS showed 95% product formation and absence of starting material, the reaction mixture was diluted with MTBE (500 mL) and water (500 mL) was added. The solid which was observed was filtered through a Buchner funnel and the filtrate was washed with saturated brine solution, dried over anhydrous sodium sulphate and concentrated under vacuum to give 5.5 g of crude product of 93.8% purity. The filtered solid (2 g) showed 99% of the desired mass in LCMS. Both materials were combined and purified by Grace column chromatography using ethyl acetate/ hexane as eluent. The compound eluted at 30% ethyl acetate: hexane to obtain two fractions. Fraction 1: 0.2 g of 99.25% LCMS purity. Fraction 2: 4.5 g of 99.8% LCMS purity. Yield=60%. ¹H-NMR (400 MHz, CDCl₃): δ [ppm] 2.49 (s, 6H), 2.54 (s, 6H), 6.12 (s, 2H).

### Synthesis of Intermediate 6

| | Qty | Vol. | | | |
|---|---|---|---|---|---|
| Reagent | (g) | (mL) | MW | mmoles | Eq. |
| Intermediate 5 | 4.5 | — | 282.5 | 15.9 | 1 |
| NBS | 5.92 | — | 177.98 | 33.3 | 2.1 |
| Acetonitrile | — | 510 | — | — | — |

A 1000 mL 3-necked round-bottomed flask equipped with a magnetic stirrer, addition funnel, nitrogen inlet and exhaust were used to perform the reaction. Intermediate 5 (4.5 g, 15.9 mmol) was combined with acetonitrile (450 mL) at room temperature (25° C.). NBS (5.92 g, 33.3 mmol) dissolved in acetonitrile (60 mL) was added slowly dropwise over a period of 20 minutes at room temperature (25° C.). The reaction mixture was stirred for 16 hours at room temperature (25° C.). Reaction monitoring by LCMS showed 99% of the desired product mass. The above reaction mixture was diluted with ethyl acetate (500 mL), then washed with water, saturated brine solution, dried over anhydrous sodium sulphate and concentrated under vacuum to get 7.2 g of crude product with 99% desired mass. The crude product was purified by column chromatography using 2 to 10% DCM in hexane as an eluent to isolate 5.5 g of 99.1% purity desired product. Yield=78%. ¹H-NMR (400 MHz, CDCl₃): δ [ppm] 2.52 (s, 6H), 2.60 (s, 6H).

### Synthesis of Monomer 2

| | Qty | Vol. | | | |
|---|---|---|---|---|---|
| Reagent | (g) | (mL) | MW | Moles | Eq. |
| Intermediate 6 | 4.15 | — | 440.32 | — | 1 |
| Ammonia in THF (0.5M) | — | 290 | — | — | — |
| Toluene | — | 290 | — | — | — |

A 1 L multi neck RBF equipped with magnetic stir bar, addition funnel and Nitrogen inlet and ice-cold water bath was used to perform the reaction. Intermediate 6 (4.15 g) was combined with toluene (290 mL). The reaction mass was cooled to 0° C., then ammonia in THF (0.5M, 290 mL) was slowly added using addition funnel over a period of 30 minutes. The reaction was continued for 2 hours at 0° C. -10° C., then the reaction mass was slowly allowed to reach room temperature (25° C.) and stirring was continued for 16 hours. Reaction progress was monitored by LCMS, which showed 96.7% desired mass along with 1.8% starting material. The reaction was stopped and some solid formation was observed. The reaction mixture was filtered through a celite plug and the filtrate was concentrated to get 1 g of crude product (Fraction 1). The celite plug was washed thoroughly with THF and 3.28 g of crude product was isolated (Fraction 2). The crude products obtained (Fraction 1 and Fraction 2) were separately recrystallized using acetonitrile/ toluene to obtain 0.63 g of 99.76% pure and 2.4 g of 98.77% pure product. 2.4 g of 98.77% pure product was again recrystallized twice using a mixture of acetonitrile (230 mL) and toluene (50 mL) to obtain 2.04 g of 99.63% pure product. Both the fractions were combined and triturated with acetonitrile (30 mL) to isolate 2.63 g of Monomer 2 with 99.8% purity. Yield=66%. ¹H-NMR (400 MHz, DMSO-d₆): δ [ppm] 2.37 (s, 6H), 2.42 (s, 6H).

### Polymer

Polymer 1 was formed by Suzuki polymerisation as described in WO 00/53656 of Monomer 1 (40 mol %) with the following monomers followed by hydrolysis of ester groups R^{10′} into cesium carboxylate groups R¹⁰ as disclosed in WO 2012/133229, the contents of which are incorporated herein by reference:

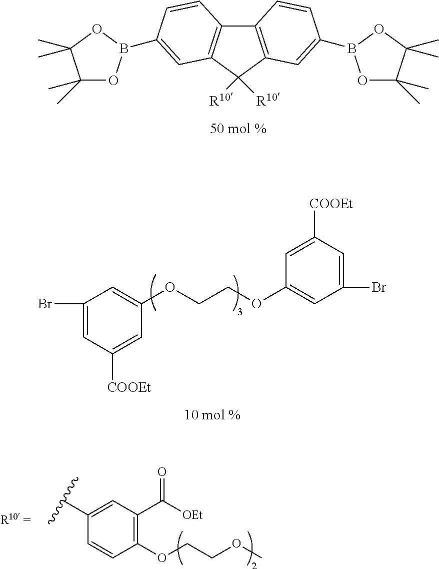

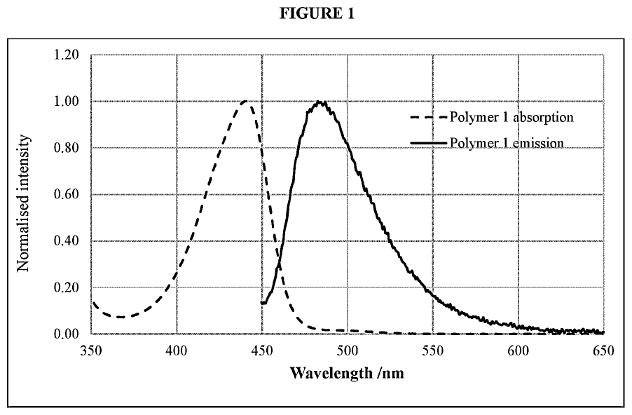

The absorption and emission spectra of Polymer 1 in methanol solution is shown in FIG. 1. The absorption and emission maxima are given in Table 1.

TABLE 1

| | | | | Stokes |
|---|---|---|---|---|
| | | λabs. / | λem / | shift/ |
| | Polymer structure | nm | nm | nm |
| Polymer 1 | 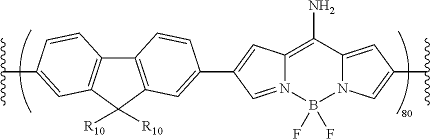 | 442 | 484 | 42 |
| | 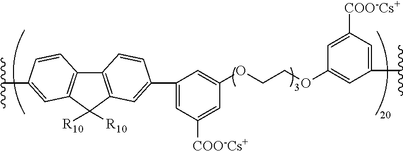 | | | |
| | 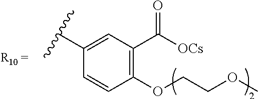 | | | |

1. A conjugated polymer comprising a repeat unit of formula (I): 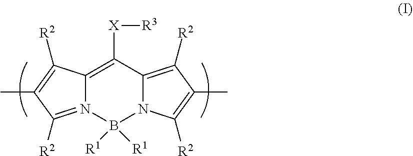
wherein:
R¹ in each occurrence is a substituent;
R² in each occurrence is independently H or a substituent;
X is selected from O, S and NR³; and
R³ in each occurrence is independently H or a substituent.

2. The conjugated polymer according to claim 1 wherein each R³ is independently selected from;
non-ionic groups selected from:
H;
C₁₋₂₀ alkyl or C₁₋₂₀ alkylene-Ar² wherein Ar² is an aryl or heteroaryl group which may be unsubstituted or substituted with one or more non-ionic substituents and one or more non-adjacent C atoms of the C₁₋₂₀ alkyl or alkylene other than a terminal C atom or a C atom bound to X may be replaced with O, S, CO or COO and one or more H atoms may be replaced with F; and
an aryl or heteroaryl group A² which is unsubstituted or substituted with one or more non-ionic substituents;
and
ionic groups.

3. (canceled)

4. The conjugated polymer according to claim 1 wherein each R¹ is selected from F, CN, C₁₋₆ alkyl, C₁₋₆ alkynyl, —(OC2H₅)u-H wherein u is 1-5 and an unsubstituted or substituted aromatic or heteroaromatic group.

5. The conjugated polymer according to claim 1 wherein X is NR³.

6. The conjugated polymer according to claim 1 wherein the conjugated polymer is a copolymer comprising the repeat unit of formula (I) and one or more co-repeat units.

7. The conjugated polymer according to claim 6 wherein the conjugated polymer comprises an arylene or heteroarylene co-repeat unit which is unsubstituted or substituted with one or more substituents.

8. The conjugated polymer according to claim 1 wherein the conjugated polymer has a photoluminescence spectrum having a peak in the range of 400-500 nm.

9. The conjugated polymer according to claim 1 wherein the conjugated polymer has a Stokes shift of less than 50 nm.

10. The conjugated polymer according to claim 1 wherein the polymer has a solubility of at least 0.1 mg/mg in at least one of water and methanol.

11. A monomer of formula (I-M): 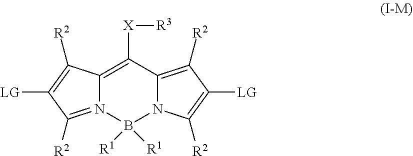
wherein:
R¹ in each occurrence is a substituent;
R² in each occurrence is independently H or a substituent;
X is selected from O, S and NR³;
R³ in each occurrence is independently H or a substituent; and
each LG is independently selected from the group consisting of boronic acid; boronic esters; halides; and pseudohalides.

12. A method of forming a conjugated polymer according to claim 1 comprising polymerisation of a monomer according to claim 11.

13. A light-emitting particle comprising the conjugated polymer according to claim 1.

14. The light-emitting particle according to claim 13 wherein the particle comprises a matrix material.

15. The light-emitting particle according to claim 14 wherein the matrix material is silica.

16. A light-emitting marker comprising the conjugated polymer or the light-emitting particle according to claim 1 and a binding group comprising a biomolecule.

17. A precursor of the light-emitting marker according to claim 16 comprising a functional group capable of attaching to the binding group.

18. A method of forming the light-emitting marker according to claim 16 comprising attaching the binding group to the functional group of the precursor.

19. A formulation comprising the conjugated polymer, light-emitting particle or precursor according to claim 1 dissolved or dispersed in one or more solvents.

20. A method of sequencing nucleic acids comprising contacting a primed template nucleic acid molecule with a polymerase and a test nucleotide;
incorporating the test nucleotide into a primed strand of the primed template only if it comprises a base complementary to the next base of the template strand;
irradiating the primed strand; and
determining from luminance of the primed strand if the test nucleotide has been incorporated into the primed strand,
wherein the test nucleotide of the irradiated primed strand is bound to a conjugated polymer, light-emitting particle or light-emitting marker according to claim 1 configured to bind to the test nucleotide.

21. A method of identifying a target analyte in a sample, the method comprising irradiating the sample to which has been added a conjugated polymer, light-emitting particle or light-emitting marker according to claim 1 configured to bind to the target analyte; and detecting emission from the conjugated polymer.

22. (canceled)
